(19) United States
(12) Patent Application Publication
(10) Pub. No.: US 2025/0093332 A1
(43) Pub. Date: Mar. 20, 2025

(54) METHODS AND RELATED ASPECTS OF QUANTIFYING PROTEIN STABILITY AND MISFOLDING

(71) Applicant: ARIZONA BOARD OF REGENTS ON BEHALF OF ARIZONA STATE UNIVERSITY, Scottsdale, AZ (US)

(72) Inventors: Kerry Geiler-Samerotte, Scottsdale, AZ (US); Yuichi Eguchi, Tempe, AZ (US); Natalie Quan, Fountain Hills, AZ (US)

(21) Appl. No.: 18/727,210

(22) PCT Filed: Jan. 10, 2023
(86) PCT No.: PCT/US2023/060420

§ 371 Date: Jul. 8, 2024

Related U.S. Application Data
(60) Provisional application No. 63/298,759, filed on Jan. 12, 2022.

(51) Int. Cl. G01N 33/50; C12M 1/34; C12M 1/36; G01N 33/68

(52) U.S. Cl. CPC G01N 33/5014; C12M 41/36; C12M 41/48; G01N 33/6803

(57) ABSTRACT

Provided herein are methods of quantifying protein folding and stability. Some embodiments provide methods of detecting a misfolded target protein that include determining a growth rate or relative fitness of a first cell population that comprises a first fusion polypeptide comprising a first variant of a target protein disposed between segments of a toxic indicator protein, which segments together induce the first cell population to grow at a lower rate when the target protein is relatively less misfolded than when the target protein is relatively more misfolded, and determining that the growth rate or fitness of the first cell population varies from a growth rate or fitness of a second cell population that comprises a second fusion polypeptide comprising a second variant of the target protein disposed between the segments of the toxic Indicator protein. Related nucleic acids, kits, and systems are also provided.

### CROSS-REFERENCE TO RELATED APPLICATIONS

This application claims priority to U.S. Provisional Patent Application Ser. No. 63/298,759, filed Jan. 12, 2022, the disclosure of which is incorporated herein by reference.

### STATEMENT OF GOVERNMENT SUPPORT

This invention was made with government support under R35 GM133674 awarded by the National Institutes of Health. The government has certain rights in the invention.

### BACKGROUND

Protein misfolding is a root cause of many biological problems. For example, it causes diseases like ALS, Parkinson's, and Alzheimer's. It is also important factor in cancer evolution, owing, at least in part, to the high mutational rates exhibited in many tumors. As an additional example, protein misfolding is also a problem in the synthetic biology and bioproducts industry, where strains created to produce bioengineered products frequently express those products at relatively low yields. In many of these cases, this inefficiency stems from bioengineered protein instability, which leads to protein misfolding.

Accordingly, there is a need for effective techniques for quantitatively detecting and measuring protein stability and misfolding.

### SUMMARY

This disclosure describes methods, systems, and related aspects for detecting and quantifying protein stability and misfolding. The methods generally Include comparing relative growth rates of cell populations having fusion polypeptides with substantially identical toxic indicator proteins and differing target protein variants. In some implementations, these methods are performed as part of massively parallel therapeutic protein candidate, or other polypeptide, screening processes. These and other aspects will be apparent upon complete review of the present disclosure, including the accompanying figures.

In one aspect, the present disclosure provides a method of detecting a misfolded target protein. The method includes determining a growth rate or relative fitness of at least a first cell population that comprises a first fusion polypeptide comprising a first variant of a target protein disposed between segments of a toxic indicator protein, which segments together induce the first cell population to grow at a lower rate when the target protein is relatively less misfolded than when the target protein is relatively more misfolded. The method also includes determining that the growth rate or fitness of the first cell population varies from a growth rate or fitness of at least a second cell population that comprises a second fusion polypeptide comprising a second variant of the target protein disposed between the segments of the toxic indicator protein, thereby detecting the misfolded target protein.

In some embodiments, the method further comprises quantifying a misfolding or stability measure of the first variant of the target protein by comparing the growth rate or fitness of the first cell population to that of at least one other cell population. In some embodiments, the first cell population growth rate or fitness is higher or lower than the growth rate or fitness of the second cell population. In some embodiments, the target protein is attached to the segments of the toxic indicator protein via a linker moiety. In some embodiments, the second variant of the target protein is a wild-type form of the target protein. In some embodiments, the first variant of the target protein comprises one or more mutations. In some embodiments, the first variant and/or the second variant of the target protein is disposed between the segments of the toxic indicator protein such that localization of the target protein is at least disrupted (e.g., cancelled or the like).

In some embodiments, the toxic indicator protein is an inducible toxic indicator protein. In some embodiments, the method further comprises exposing the inducible toxic indicator protein to an inducing agent. In some embodiments, the inducible toxic indicator protein comprises an FCY1 protein and wherein the method further comprises contacting the first cell population with 5-fluorocytosine (5FC) prior to and/or when determining the growth rates of the first and second cell populations.

In some embodiments, the method comprises determining growth rates of multiple cell populations substantially in parallel with one another, wherein two or more of the multiple cell populations comprise different fusion polypeptides comprising different variants of the target protein disposed between the segments of the toxic indicator protein, and determining whether the growth rates or fitness of multiple cell populations vary from to that of at least one other cell population. In some embodiments, the different fusion polypeptides comprise about 3, about 4, about 5, about 6, about 7, about 8, about 9, about 10, about 20, about 30, about 40, about 50, about 60, about 70, about 80, about 90, about 100, about 200, about 300, about 400, about 500, about 600, about 700, about 800, about 900, about 1000, about 2000, about 3000, about 4000, about 5000, or more different variants of the target protein.

In some embodiments, the method further comprises generating nucleic acid variants that encode the different variants of the target protein. In some embodiments, the method further comprises expressing the first and second fusion polypeptides from nucleic acid plasmids disposed in the first and second cell populations, which nucleic acid plasmids encode the first or the second fusion polypeptides. In some embodiments, the nucleic acid plasmids comprise one or more nucleic acid barcodes that distinguish a nucleic acid plasmid encoding the first fusion polypeptide from a nucleic acid plasmid encoding the second fusion polypeptide. In some embodiments, wherein at least one of the nucleic acid barcodes comprises a randomly selected sequence of nucleotides. In some embodiments, the nucleic acid barcodes comprise donor and/or guide nucleic acid sequences and wherein the method further comprises using the nucleic acid barcodes that comprise the donor and/or the guide nucleic acid sequences to generate one or more mutations in a nucleic acid that encodes the target protein. In some embodiments, the method comprises pooling the first and second cell populations in a container. In some embodiments, the method comprises determining the growth rate or relative fitness of the first cell population and the growth rate or relative fitness of the second cell population from changes in nucleic acid barcode frequencies observed over time (e.g., by sequencing nucleic acid plasmids from the cell populations at various time points or the like).

In some embodiments, the target protein comprises a disease-associated protein. In some embodiments, the target protein comprises a candidate therapeutic protein. In some embodiments, the target protein comprises a recombinantly engineered protein.

In another aspect, the present disclosure provides a nucleic acid plasmid (e.g., comprising a pWF5 plasmid (e.g., pRS315 plasmid backbone, etc.) or a p6F5 plasmid, among many others) that includes a nucleotide sequence that encodes a fusion polypeptide comprising a variant of a target protein disposed between segments of a toxic indicator protein, wherein upon expression of the fusion polypeptide in a cell population, the segments together induce the cell population to grow at a lower rate when the target protein is relatively less misfolded than when the target protein relatively more misfolded.

In some embodiments, the nucleic acid plasmid further includes additional nucleotide sequences disposed between the segments of the toxic indicator protein and the variant of the target protein, which additional nucleotide sequences encode polypeptide linker moleties. In some embodiments, the variant of the target protein is a wild-type form of the target protein. In some embodiments, the variant of the target protein comprises one or more mutations.

In some embodiments, the toxic indicator protein is an inducible toxic indicator protein. In some embodiments, the inducible toxic indicator protein comprises an FCY1 protein. In some embodiments, the target protein comprises a disease-associated protein. In some embodiments, the target protein comprises a candidate therapeutic protein. In some embodiments, the target protein comprises a recombinantly engineered protein. In some embodiments, the variant of the target protein is disposed between the segments of the toxic indicator protein such that localization of the target protein is at least disrupted upon expression of the fusion polypeptide in the cell population. In some embodiments, a cell population comprises the nucleic acid plasmid. In some embodiments, a kit comprises the nucleic acid plasmid.

In another aspect, the present disclosure provides a system that includes a sample container positioning area that comprises a sample container that comprises at least a first cell population that comprises a first fusion polypeptide comprising a first variant of a target protein disposed between segments of a toxic indicator protein, which segments together induce the first cell population to grow at a lower rate when the target protein is relatively less misfolded than when the target protein relatively more misfolded. In some embodiments, the inducible toxic indicator protein comprises an FCY1 protein. The system also includes a detector configured to detect a growth rate or fitness of the first cell population. In addition, the system also includes a controller that comprises, or is capable of accessing, computer readable media comprising non-transitory computer-executable instructions which, when executed by at least one electronic processor, perform at least: determining whether the growth rate or fitness of the first cell population varies from a growth rate or fitness of at least a second cell population that comprises a second fusion polypeptide comprising a second variant of the target protein disposed between the segments of the toxic indicator protein.

In some embodiments, the first fusion polypeptide is expressed from nucleic acid plasmids disposed in the first cell population. In some of these embodiments, the nucleic acid plasmids encode the first fusion polypeptide and comprise one or more nucleic acid barcodes that distinguish the nucleic acid plasmid encoding the first fusion polypeptide from a nucleic acid plasmid encoding the second fusion polypeptide. In some of these embodiments, the detector comprises a nucleic acid sequencing device that generates sequence information from the nucleic acid plasmids. In some of these embodiments, the non-transitory computer-executable instructions which, when executed by the electronic processor, further perform determining the growth rate of the first cell population and the growth rate of the second cell population from changes in nucleic acid barcode frequencies observed over time in the sequence information. In some embodiments, the first fusion polypeptide is expressed from a set of first nucleic acid plasmids disposed in the first cell population, which first nucleic acid plasmids encode the first fusion polypeptide, and wherein a set of second nucleic acid plasmids disposed in the first cell population comprise one or more nucleic acid barcodes that distinguish the first nucleic acid plasmid encoding the first fusion polypeptide from a third nucleic acid plasmid encoding the second fusion polypeptide, wherein the detector comprises a nucleic acid sequencing device that generates sequence information from the nucleic acid plasmids, and wherein the non-transitory computer-executable instructions which, when executed by the electronic processor, further perform determining the growth rate of the first cell population and the growth rate of the second cell population from changes in nucleic acid barcode frequencies observed over time in the sequence information. In some embodiments, at least one of the nucleic acid barcodes comprises a randomly selected sequence of nucleotides. In some embodiments, the nucleic acid barcodes comprise donor and/or guide nucleic acid sequences that are used to generate one or more mutations in a nucleic acid that encodes the target protein. In some embodiments, wherein the first variant and/or the second variant of the target protein is disposed between the segments of the toxic indicator protein such that localization of the target protein is at least disrupted.

### BRIEF DESCRIPTION OF DRAWINGS

FIG. 1 is a flow chart that schematically shows exemplary method steps of detecting a misfolded target protein according to some aspects disclosed herein.

FIG. 2 schematically depicts an exemplary pWF5-TYP (8053 bp) DNA plasmid map according to some aspects disclosed herein.

FIG. 3 is a schematic diagram of an exemplary system suitable for use with certain aspects disclosed herein.

FIGS. 4A-4E depict aspects of experiments showing varied concentrations of 5-fluorocytosine (5FC) and other data. (A) Schematic of a pWF5-YFP plasmid map. (B) 5FC conditions in fcy1Δ strain. (C) Plot of maximum growth rate (Y-axis) versus 5FC concentration (nM) (X-axis). (D) Histogram showing the maximum growth rate (Y-axis) versus YFP protein variant (X-axis). (E) Histogram showing the ratio of maximum growth rate (MGR) (Y-axis) versus YFP protein variant (X-axis).

FIGS. 5A-5D depict aspects of experiments involving modified green fluorescent protein (GFP). (A) The plasmid that was used in measuring the localization pattern of modified GFPs when they are sandwiched inside the Intra-FCY1 toxic indicator protein. Fcy1-fused modified GFP was expressed from the tunable TetO-7.1 promoter on a single-copy plasmid (pRS315). Typically, these modified GFPs localize to the mitochondria, endoplasmic reticulum, or peroxisome. (B) Micrographs of cells expressing Fcy1-fused modified GFPs or modified GFPs when they are not fused to Fcy-1 demonstrating that sandwiching within the Foy-1 protein cancels location to organelles and allows the target protein to be expressed in the cytosol. (C) Maximum growth rate of the yeast cells harboring the plasmids expressing Fcy1-Mito-GFP, Fcy1-ER-GFP, and Fcy1-Pero-GFP at aTc 500 nM in 10 mM 5-FC and 0 mM 5-FC conditions. This is further evidence that the Fcy-1 sandwich cancels organelle localization and allows expression in the cytosol where the Fcy-1 protein can reduce growth rate when expressed in media containing 10 mM 5-FC. (D) Ratio of the maximum growth rate of the yeast cells harboring the plasmids expressing Fcy1-Mito-GFP, Fcy1-ER-GFP and Fcy1-Pero-GFP calculated from the results of (C). The decreased growth of the GFP and YFP targets when fused to Fcy-1 relative to the empty vector control is further evidence that the organelle localization of the modified GFP variants has been cancelled.

FIGS. 6A-6C show how barcode frequencies change over time for about 200 different barcodes. Each barcode tracks the frequency of a strain containing a plasmid bearing a different variant of YFP sandwiched between two halves of the toxic indicator Fcy-1 protein. All 200 strains are mixed together in the same vessel and allowed to grow for 9 days. The horizontal axes show time and ‘t1’ represents day one, while ‘t9’ represents day 9. The vertical axis shows the frequency of each barcode. Each line represents a different barcode. The dashed lines show barcodes that represent strains possessing a YFP that is suspected to misfold. These strains have barcodes that stay at roughly the same frequency over time when the YFP-Fcy1 fusion protein is not expressed (0 nM aTc; panel A). But these barcodes increase in frequency when the fusion protein is expressed (500 nM aTc) and when 5FC is added to the media (panels B and C). This is evidence that the method of this disclosure is effective at detecting protein misfolding in high-throughput experiments.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Barcode: As used herein, “barcode” in the context of nucleic acids refers to a nucleic acid molecule comprising a sequence that can serve as a molecular identifier. For example, individual “barcode” sequences are typically added to each DNA fragment during next-generation sequencing (NGS) library preparation so that each read can be identified and sorted before the final data analysis. In the current disclosure, individual “barcode” sequences may be present within the nucleic acid plasmid that comprises the target protein disposed between the segments of the toxic indicator protein. In other embodiments, barcodes may also be present on another plasmid. The key is that DNA “barcodes” are sequences of DNA used to identify different cells, strains, or experiments.

Cell: As used herein, the phrase “cell” or “host cell” refers to a cell into which exogenous DNA (recombinant or otherwise) has been Introduced. For example, host cells may be used to produce the fusion polypeptides referenced herein by standard production techniques. Persons of skill upon reading this disclosure will understand that such terms refer not only to the particular subject cell, but, to the progeny of such a cell. Because certain modifications may occur in succeeding generations due to either mutation or environmental influences, such progeny may not, in fact, be identical to the parent cell, but are still included within the scope of the term “cell” or “host cell” as used herein. In some embodiments, host cells include any prokaryotic and eukaryotic cells suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: Chinese Hamster Ovary or CHO cells (e.g., CHO K1, DXB-11 CHO, Veggie-CHO), COS cells (e.g., COS-7), retinal cells, Vero cells, CV1 cells, kidney cells (e.g., HEK293, 293 EBNA, MSR 293, MDCK, HaK, BHK), Hela cells, HepG2 cells, W138 cells, MRC 5 cells, Colo205 cells, HB 8065 cells, HL-60 cells, BHK21 cells, Jurkat cells, Daudi cells, A431 (epidermal) cells, CV-1 cells, U937 cells, 3T3 cells, L cells, C127 cells, SP2/0 cells, NS-0 cells, MMT 060562 cells, Sertoli cells, BRL 3A cells, HT1080 cells, myeloma cells, tumor cells, and a cell line derived from an aforementioned cell. In some embodiments, the cell comprises one or more viral genes, e.g., a retinal cell that expresses a viral gene (e.g., a PER.C6™ cell).

Detect: As used herein, “detect,” “detecting,” or “detection” refers to an act of determining the existence or presence of one or more analytes (e.g., misfolded target proteins) in a given sample.

Encoding: As used herein, “encoding” or “encode” refers to i) genetic information comprised in a DNA sequence that can be transcribed into an mRNA molecule, and/or ii) genetic information comprised in an mRNA molecule that can be translated into an amino acid sequence. Hence, these terms also cover genetic information comprised in the DNA that can be converted via transcription of an mRNA molecule into an amino acid sequence such as a protein.

Expression: The term “expression”, when used in reference to a nucleic acid herein, refers to one or more of the following events: (1) production of an RNA transcript of a DNA template (e.g., by transcription); (2) processing of an RNA transcript (e.g., by splicing, editing, 5′ cap formation, and/or 3′ end formation); (3) translation of an RNA into a polypeptide; and/or (4) post-translational modification of a polypeptide.

Fitness: As used herein, the term “fitness” in the context of cell population comparisons refers to one or more cell populations that exhibit at least one measurable feature that has a higher measured value than that exhibited by one or more other cell populations. In some embodiments, for example, the measurable features comprise relative growth rates measured for the cell populations being compared to one another.

In some embodiments: As used herein, the term “in some embodiments” refers to embodiments of all aspects of the disclosure, unless the context clearly indicates otherwise.

Misfolded: As used herein, “misfolded” in the context of polypeptides refers to polypeptides that have formed incorrect three-dimensional structures that result in inactive polypeptides or polypeptides that modified or toxic functionality.

Mutation: As used herein, “mutation,” “nucleic acid variant,” “variant,” or “genetic aberration” refers to a variation from a known reference sequence and includes mutations such as, for example, single nucleotide variants (SNVs), copy number variants or variations (CNVs)/aberrations, insertions or deletions (indels), truncation, gene fusions, transversions, translocations, frame shifts, duplications, repeat expansions, and epigenetic variants. A mutation can be a germline or somatic mutation. In some embodiments, a reference sequence for purposes of comparison is a wildtype genomic sequence of the species of the subject providing a test sample, typically the human genome.

Nucleic Acid: As used herein, “nucleic acid” refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., bromodeoxyuridine (BrdU)), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA, cfDNA, ctDNA, or any combination thereof.

Plasmid: As used herein, “plasmid” refers to a vector comprising a double-stranded DNA molecule. If not stated otherwise, the term “plasmid” refers to a circular DNA molecule, though the term can also encompass linear DNA molecules. In particular, the term “plasmid” also covers molecules which result from linearizing a circular plasmid by cutting it, e.g. with a restriction enzyme, thereby converting the circular plasmid molecule into a linear molecule. Plasmids can replicate, that is, amplify in a cell independently from the genetic information stored as chromosomal DNA in the cell and can be used for cloning, that is, for amplifying genetic information in a cell. In some embodiments, a DNA plasmid of the present disclosure is a medium- or high-copy plasmid. Examples for such high-copy plasmids are vectors based on pUC, pBluescript®, pGEMO, pTZ plasmids or any other plasmids which contain an origin of replication (e.g., pMB1, pColE1) that support high copies of the plasmid.

Protein: As used herein, “protein” or “polypeptide” refers to a polymer of at least two amino acids attached to one another by a peptide bond. Examples of proteins include enzymes, hormones, antibodies, and fragments thereof.

Recombinant: As used herein, the term “recombinant” in the context of polypeptides is intended to refer to polypeptides (e.g., fusion polypeptides as described herein) that are designed, engineered, prepared, expressed, created or isolated by recombinant means, such as polypeptides expressed using a recombinant expression vector transfected into a host cell, polypeptides isolated from a recombinant, combinatorial polypeptide library or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source. In some embodiments, one or more such selected sequence elements results from the combination of multiple (e.g., two or more) known sequence elements that are not naturally present in the same polypeptide.

Sequencing: As used herein, “sequencing” refers to any of a number of technologies used to determine the sequence (e.g., the identity and order of monomer units) of a biomolecule, e.g., a nucleic acid such as DNA or RNA. Exemplary sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion POR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLID™ sequencing, MS-PET sequencing, and a combination thereof. In some embodiments, sequencing can be performer by a gene analyzer such as, for example, gene analyzers commercially available from Illumina, Inc., Pacific Biosciences, Inc., or Applied Biosystems/Thermo Fisher Scientific, among many others.

Sequence Information: As used herein, “sequence information” in the context of a nucleic acid polymer means the order and identity of monomer units (e.g., nucleotides, etc.) in that polymer.

Toxic Indicator Protein: As used herein, “toxic indicator protein” refers to a protein that influences the growth rate of a given cell when that protein is present in the cell in a catalytically active form. In some embodiments, the fusion polypeptides disclosed herein include segments of a toxic indicator protein, which segments together induce the first cell population to grow at a lower rate when a given target protein is relatively less misfolded (and hence, the segments of the toxic indicator protein are disposed relative to one another such that the toxic indicator protein is functional (e.g., catalytically active)) than when the target protein relatively more misfolded (and hence, the segments of the toxic indicator protein are disposed relative to one another such that the toxic indicator protein is non-functional or has reduced functionality). Essentially any toxic indicator protein is optionally used in the fusion polypeptides disclosed herein. In some embodiments, for example, toxic Indicator proteins used in the fusion polypeptides are FCY1 (cytosine deaminase) proteins (EC: 3.5.4.1) encoded by an FCY1 gene, which proteins catalyze the hydrolytic deamination of cytosine to uracil, 5-methylcytosine to thymine, or 5-fluorocytosine (5FC) to form the anticancer drug 5-fluorouracil (5FU). In some embodiments, 5FC is use as an inducing agent to induce the activity of FCY1 proteins in the fusion polypeptides of the present disclose.

Wild-type (WT): As is understood in the art, the term “wild-type” generally refers to a normal form of a protein or nucleic acid, as is found in nature.

### DETAILED DESCRIPTION

Protein misfolding happens within cells constantly, and there is increasing interest in understanding the basics of protein misfolding mechanisms. Protein misfolding can cause disease, such as ALS, Parkinson's, and Alzheimer's, can inhibit the efficiency levels of synthetic biologic creation in the bioproduction industry, and may even be used as a weapon against cancer cells by inducing misfolding of key proteins. Accordingly, in some aspects, the present disclosure provide methods of detecting and quantifying misfolded proteins. In some embodiments, the methods are used in massively parallel formats to screen the folding/misfolding of thousands of mutant versions of target proteins created using CRISPR or another technique. These mutant versions are typically encoded in DNA plasmids that include tunable promoters to dial protein expression up or down. Typically, these protein variants are encoded in plasmids as part of fusion polypeptide constructs that also include inducible toxic indicator proteins that are used to detect the folding status of the target proteins expressed in host cells from the plasmids. In some implementations, the technology presented herein provides ways to study proteins of interest, analyze variants, and quantify their stability and toxicity in order to identify misfolding causing mutations within the selected proteins. These and other aspects will be apparent upon complete review of the present disclosure, including the accompanying figures.

In some embodiments, the methods and other aspects of the present disclosure enable the massive parallel quantification of thousands of mutant proteins, for example, as part of drug candidate screening applications. Typically, an inducible toxic protein is bifurcated with the protein of interest in a fusion polypeptide construct. If the protein of interest variant misfolds it will generally impact the toxic indicator protein too, thus inhibiting its effects or activity. This can be quantitatively detected, as the more and faster the cells grow, the more misfolded the target protein is (as it is affecting the toxic protein and inhibiting toxicity). The methods can be used to compare thousands of mutants and devise which ones cause the most misfolding. Some embodiments enable the measurement of relative growth rates by culturing the mutants in the same flask or other sample container and sampling for each mutant's specific “DNA Barcode” and noting changes in frequency over time. Typically, DNA barcodes are detected using next generation sequencing. In some embodiments, inducible toxic indicator proteins are non-toxic unless a drug or other inducing additive is mixed into the media. In some of these embodiments, for example, toxic protein FCY1 is used along with the inducer drug 5-fluorocytosine (5FC).

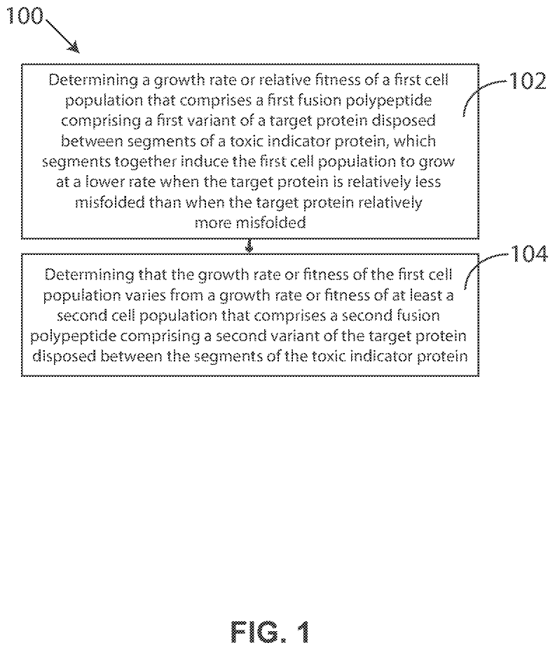

To illustrate, FIG. 1 is a flow chart that schematically shows exemplary method steps of detecting a misfolded target protein. As shown, method 100 includes determining a growth rate or relative fitness of at least a first cell population (e.g., S. cerevisiae host cells or the like) that comprises a first fusion polypeptide comprising a first variant of a target protein disposed between segments of a toxic indicator protein, which segments together induce the first cell population to grow at a lower rate when the target protein is relatively less misfolded than when the target protein relatively more misfolded (step 102). Method 100 also includes determining that the growth rate or fitness of the first cell population varies from a growth rate or fitness of at least a second cell population that comprises a second fusion polypeptide comprising a second variant of the target protein disposed between the segments of the toxic indicator protein (step 104). Essentially any target protein is optionally used. In some embodiments, for example, SOD1, αSYN, or YFP mutants are used.

In some embodiments, method 100 further comprises quantifying a misfolding or stability measure of the first variant of the target protein from the growth rate of the first cell population. In some embodiments, the first cell population growth rate is higher than the growth rate of the second cell population. In some embodiments, the target protein is attached to the segments of the toxic indicator protein via a linker moiety. In some embodiments, the second variant of the target protein is a wild-type form of the target protein. In some embodiments, the first variant of the target protein comprises one or more mutations.

In some embodiments, the toxic indicator protein is an inducible toxic indicator protein. In some embodiments, method 100 further comprises exposing the inducible toxic indicator protein to an inducing agent. In some embodiments, the inducible toxic indicator protein comprises an FCY1 protein and method 100 further comprises contacting the first cell population with 5-fluorocytosine (5FC) prior to and/or when determining the growth rates of the first and second cell populations.

In some embodiments, method 100 comprises determining growth rates of multiple cell populations substantially in parallel with one another, wherein two or more of the multiple cell populations comprise different fusion polypeptides comprising different variants of the target protein disposed between the segments of the toxic indicator protein, and determining whether the growth rates of multiple cell populations vary from the growth rate of the second cell population. In some embodiments, the different fusion polypeptides comprise about 3, about 4, about 5, about 6, about 7, about 8, about 9, about 10, about 20, about 30, about 40, about 50, about 60, about 70, about 80, about 90, about 100, about 200, about 300, about 400, about 500, about 600, about 700, about 800, about 900, about 1000, about 2000, about 3000, about 4000, about 5000, or more different variants of the target protein. In some embodiments, CRISPEY (an ultra-high efficiency CRISPR method) is used to create yeast strains, each of which possesses an engineered protein variant and a unique DNA barcode associated with that mutation.

In some embodiments, method 100 further comprises generating nucleic acid variants that encode the different variants of the target protein. In some embodiments, method 100 further comprises expressing the first and second fusion polypeptides from nucleic acid plasmids disposed in the first and second cell populations, which nucleic acid plasmids encode the first or the second fusion polypeptides. In some embodiments, the nucleic acid plasmids comprise one or more nucleic acid barcodes that distinguish a nucleic acid plasmid encoding the first fusion polypeptide from a nucleic acid plasmid encoding the second fusion polypeptide. In some embodiments, method 100 comprises pooling the first and second cell populations in a container. In some embodiments, method 100 comprises determining the growth rate of the first cell population and the growth rate of the second cell population from changes in nucleic acid barcode frequencies observed over time (e.g., by sequencing nucleic acid plasmids from the cell populations at various time points or the like).

In some embodiments, the target protein comprises a disease-associated protein. In some embodiments, the target protein comprises a candidate therapeutic protein. In some embodiments, the target protein comprises a recombinantly engineered protein.

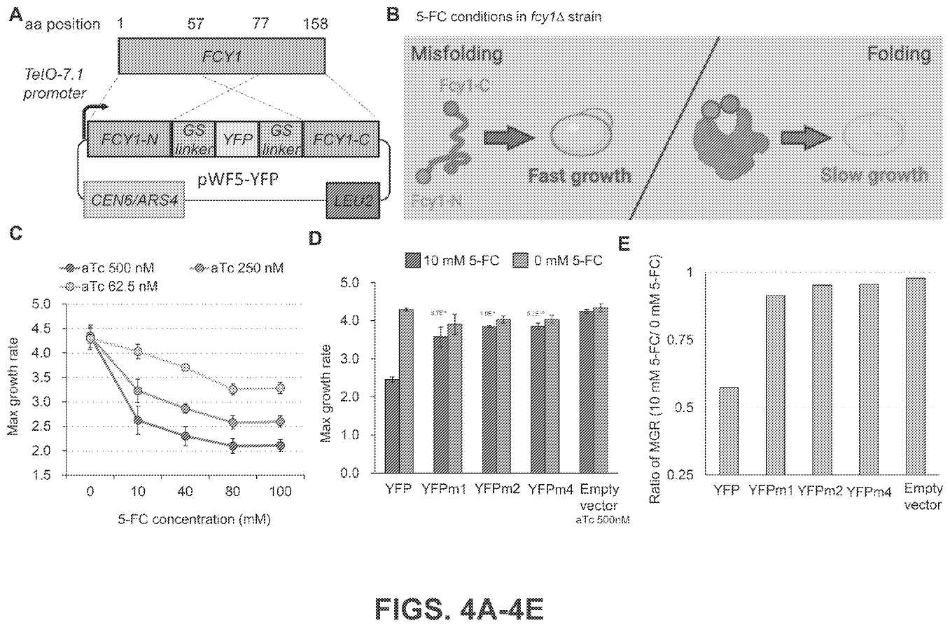

Various techniques for quantifying how mutations affect misfolding have been utilized. In some applications, for example, Western blotting of the soluble versus insoluble cell fractions is used as a gold standard for estimating protein stability. Optionally, a higher throughput method involves creating a chimeric protein by inserting the mutant protein into DHFR (an essential protein), such that cell growth declines in proportion to how much DHFR is made unstable by the misfolded protein. This system generally has a limited range because moderately and severely misfolded proteins destabilize DHFR enough to cause major growth defects. The methods and related aspects disclosed herein improve upon systems, such as the DHFR system, because, for example, they have a broader range to detect misfolded proteins than these earlier approaches, among other attributes. FIG. 4E illustrates this wider or broader range by showing that the methods and related aspects of the present disclosure can distinguish between extremely misfolded proteins, like YFP m2 and YFP m4.

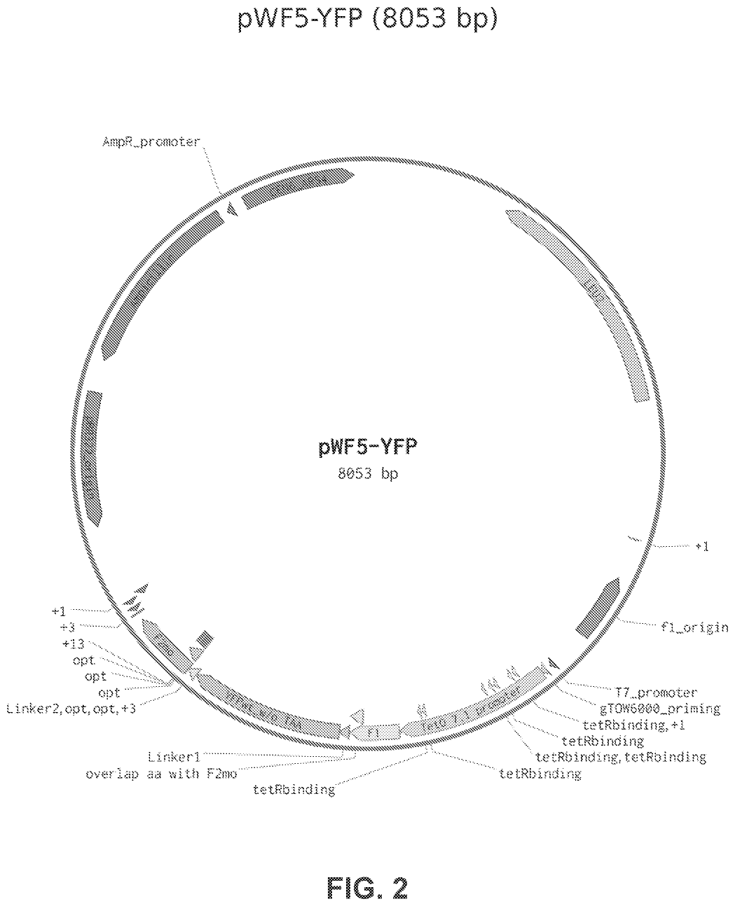

In another aspect, the present disclosure provides a nucleic acid plasmid (e.g., comprising a pWF5 plasmid (e.g., pRS315 plasmid backbone, etc.) or a p6F5 plasmid, among many others) that includes a nucleotide sequence that encodes a fusion polypeptide comprising a variant of a target protein disposed between segments of a toxic indicator protein, wherein upon expression of the fusion polypeptide in a cell population, the segments together induce the cell population to grow at a lower rate when the target protein is relatively less misfolded than when the target protein relatively more misfolded. FIG. 2 schematically depicts an exemplary pWF5-TYP (8053 bp) DNA plasmid map according to some aspects disclosed herein.

In some embodiments, the nucleic acid plasmid further includes additional nucleotide sequences disposed between the segments of the toxic indicator protein and the variant of the target protein, which additional nucleotide sequences encode polypeptide linker moieties. In some embodiments, the variant of the target protein is a wild-type form of the target protein. In some embodiments, the variant of the target protein comprises one or more mutations.

In some embodiments, the toxic indicator protein is an inducible toxic indicator protein. In some embodiments, the inducible toxic indicator protein comprises an FCY1 protein. In some embodiments, the target protein comprises a disease-associated protein. In some embodiments, the target protein comprises a candidate therapeutic protein. In some embodiments, the target protein comprises a recombinantly engineered protein. In some embodiments, a cell population comprises the nucleic acid plasmid. In some embodiments, a kit comprises the nucleic acid plasmid.

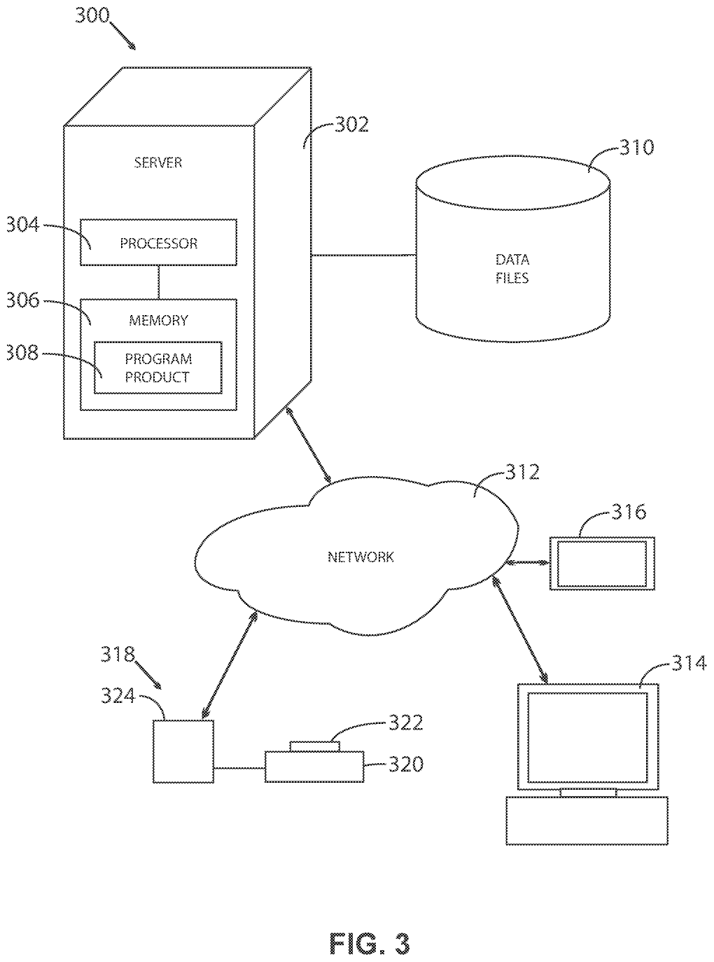

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 3 provides a schematic diagram of an exemplary system suitable for use with implementing at least aspects of the methods disclosed in this application. As shown, system 300 includes at least one controller or computer, e.g., server 302 (e.g., a search engine server), which includes processor 304 and memory, storage device, or memory component 306, and one or more other communication devices 314, 316, (e.g., client-side computer terminals, telephones, tablets, laptops, other mobile devices, etc. (e.g., for receiving protein folding/misfolding data sets, etc.) in communication with the remote server 302, through electronic communication network 312, such as the Internet or other internetwork. Communication devices 314, 316 typically include an electronic display (e.g., an internet enabled computer or the like) in communication with, e.g., server 302 computer over network 312 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 300 also includes program product 308 (e.g., for detecting misfolded target proteins as described herein) stored on a computer or machine readable medium, such as, for example, one or more of various types of memory, such as memory 306 of server 302, that is readable by the server 302, to facilitate, for example, a guided search application or other executable by one or more other communication devices, such as 314 (schematically shown as a desktop or personal computer). In some aspects, system 300 optionally also includes at least one database server, such as, for example, server 310 associated with an online website having data stored thereon (e.g., entries corresponding to protein folding/misfolding data sets, etc.) searchable either directly or through search engine server 302. System 300 optionally also includes one or more other servers positioned remotely from server 302, each of which are optionally associated with one or more database servers 310 located remotely or located local to each of the other servers. The other servers can beneficially provide service to geographically remote users and enhance geographically distributed operations.

As understood by those of ordinary skill in the art, memory 306 of the server 302 optionally includes volatile and/or nonvolatile memory including, for example, RAM, ROM, and magnetic or optical disks, among others. It is also understood by those of ordinary skill in the art that although illustrated as a single server, the illustrated configuration of server 302 is given only by way of example and that other types of servers or computers configured according to various other methodologies or architectures can also be used. Server 302 shown schematically in FIG. 3, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 300. As also understood by those of ordinary skill in the art, other user communication devices 314, 316 in these aspects, for example, can be a laptop, desktop, tablet, personal digital assistant (PDA), cell phone, server, or other types of computers. As known and understood by those of ordinary skill in the art, network 312 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 308 is optionally in the form of microcode, programs, cloud computing format, routines, and/or symbolic languages that provide one or more sets of ordered operations that control the functioning of the hardware and direct its operation. Program product 308, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 308 implementing the functionality or processes of various aspects of the present disclosure, for example, for reading by a computer. A “computer-readable medium” or “machine-readable medium” may take many forms, Including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 308 is optionally copied from the computer-readable medium to a hard disk or a similar intermediate storage medium. When program product 308, or portions thereof, are to be run, it is optionally loaded from their distribution medium, their intermediate storage medium, or the like into the execution memory of one or more computers, configuring the computer(s) to act in accordance with the functionality or method of various aspects disclosed herein. All such operations are well known to those of ordinary skill in the art of, for example, computer systems.

In some aspects, program product 308 includes non-transitory computer-executable instructions which, when executed by electronic processor 304, perform at least: determining whether the growth rate of the first cell population varies from a growth rate of at least a second cell population that comprises a second fusion polypeptide comprising a second variant of the target protein disposed between the segments of the toxic indicator protein.

Typically, misfolded target protein is detected using device 318. As shown, device 318 includes sample container positioning area 320 that comprises sample container 322 (e.g., a microplate or the like) that comprises a first cell population that comprises a first fusion polypeptide comprising a first variant of a target protein disposed between segments of a toxic indicator protein, which segments together induce the first cell population to grow at a lower rate when the target protein is relatively less misfolded than when the target protein relatively more misfolded. Device 318 also includes detector 324 configured to detect a growth rate of the first cell population. In some embodiments, for example, a determination as to which proteins are misfolding is inferred based on the relative rates at which barcode frequencies rise or fall in a population as detected using detector 324. In some embodiments, growth rates of individual strains are detected using detector 324 configured for OD monitoring or another suitable analytical technique.

### EXAMPLES

### Example 1

The present disclosure builds off the idea that one can study a protein's stability by sandwiching that protein between two halves of an essential protein. If a protein of interest misfolds, it drags the essential protein down with it. This is useful in some ways, but problematic in that the misfolded proteins end up being deadly, causing destruction of an essential protein. This makes it hard to precisely compare which proteins are more misfolded than others (since all are simply inactive or close to being inactive). The present example used a different approach in which a toxic protein (FCY1) (FIG. 4A), in lieu of an essential protein, was bifurcated with a misfolded protein. In this case, the more a protein of interest misfolds, the faster cells grow (FIG. 4B). This system can be used to compare thousands of mutant proteins to understand which mutants cause the most misfolding.

The present disclosure also illustrates the idea that relative growth rate can be measured by competing thousands of mutants in the same flask or culture vessel, and sampling how each mutant's “DNA barcode” changes in frequency over time. This example used CRISPR to create 2000 mutant versions of SOD1, and Inserted each one into the toxic FCY1 plasmid system described herein. By comparing changes in DNA barcode frequencies over time, the relative growth rates of each mutant can be compared to make inferences about mutations effect on folding. These inferences can be confirmed using Western Blots.

The example in FIG. 4 shows data obtained for a set of 4 model misfolded proteins and illustrates that this method is effective at determining protein stability (see, FIGS. 4C, 4D and 4E). In particular, these 4 model proteins included a control (YFPwt) and 3 increasingly misfolded proteins (YFPm1, YFPm2, and YFPm4). The results include the ideal conditions/concentrations at which to express each protein (FIG. 4C), as well as confirmation that the most stable protein (YFPwt) grows more slowly than the misfolded proteins (YFPm1, YFPm2, and YFPm4) in the presence of 5FC (FIGS. 4D and 4E). In the case of this example, rather than combining strains and quantifying growth rates by monitoring DNA barcode frequencies over time, each mutant strain was grown independently and its growth rate by inferred by monitoring optical density over time. Both methods (monitoring barcode frequency in mixed culture as well as monitoring OD in separate cultures) can be used in combination with the invention disclosed here. The former is higher throughput.

### Strain, Growth Conditions, and Yeast Transformation

C5W4 fcy1Δ (MATa his3Δ1::pGAL1-GAL10-SpCas9_pGAL1-GAL10-Ec86-RT_HIS3 leu2Δ0 met15Δ0::pRNR2-TetR-NLS-TUP1_ptetO7.1-TetR-NLS_MET15 ura3Δ0 fcy1Δ::HphMX6) was used in the experiment. The strain was derived from a BY4741 background (Mata his3Δ1 leu2Δ0 met15Δ0 ura3Δ0) integrated with pZS157 plasmid and P2374. Yeast culture and transformation were performed as previously described. A synthetic complete (SC) medium without leucine (Leu) with Anhydrotetracycline (aTc) and 5-FC concentrations were used for yeast culture.

### Plasmids

The plasmids in the experiment are listed in Table 1. The plasmids contain FCY1 fused YFP, YFPm1, YFPm2, or YFPm4. YFPm1, YFPm2, and YFPm4 are misfolded YFP variants. The FCY1 fusion construct consists of ptetO7.1, which regulates the expression of an FCY1 fusion protein, FCY1 N from residues 1 to 77 of yeast FCY1, FCY1 C from residues 57 to 158 of that, and YFP or YFPm1, YFPm2, and YFPm4 flanked by FCY1 N and FCY1 C with glycine-serine (GS) linkers. The plasmids were constructed by NEBuilder HiFi DNA Assembly and their sequence was verified by Sanger sequencing.

TABLE 1

| Plasmid | | Plasmid | |
|---|---|---|---|
| name | Content | backbone | Source |
| pWF5_YFP | ptetO7.1-FCY1 | pRS315 | this work |
| | N-YFP-FCY1 C | | |
| pWF5_YFPm1 | ptetO7.1-FCY1 | pRS315 | this work |
| | N-YFPm1-FCY1 C | | |
| pWF5_YFPm2 | ptetO7.1-FCY1 | pRS315 | this work |
| | N-YFPm2-FCY1 C | | |
| pWF5_YFPm4 | ptetO7.1-FCY1 | pRS315 | this work |
| | N-YFPm4-FCY1 C | | |

### Measuring Growth Rate

In FIG. 4, cellular growth was measured by monitoring OD595 every 30 minutes using an Epoch 2 Microplate spectrophotometer (BioTek). The maximum growth rate (MGR) was calculated as described previously. Average values, SD, and p-values of Welch's t-test were calculated from biological triplicates.

### Example 2

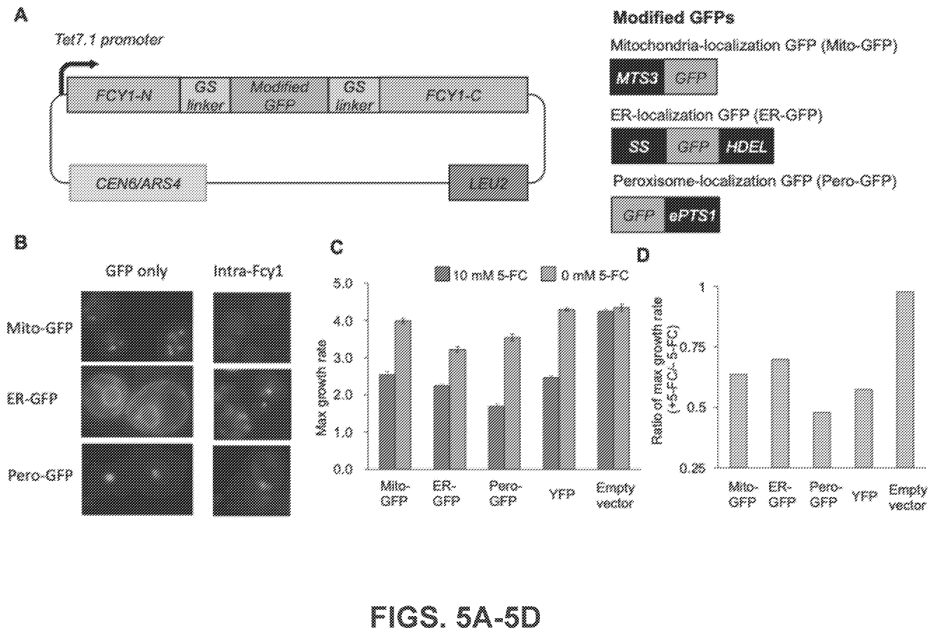

In some embodiments, the present disclosure builds off the idea that one can study a protein's stability by anchoring it to another indicator protein that affects growth rate. This approach can fail when the target protein localizes to a location in the cell that neutralizes the effect of the indicator protein on growth. The present example took the approach of sandwiching the target protein in between two halves of the indicator protein (FIG. 5A). This prevents the target protein from localizing to its usual location (FIG. 5B) and causes it to remain in the cytosol where the indicator protein has an effect on growth (FIG. 5C and FIG. 5D).

In the case of this example, rather than combining strains and quantifying growth rates by monitoring DNA barcode frequencies over time, each mutant strain was grown independently and its growth rate by inferred by monitoring optical density over time. Both methods (monitoring barcode frequency in mixed culture as well as monitoring OD in separate cultures) can be used in combination with other embodiments disclosed herein. The former is higher throughput.

The plasmids, strains and media used in this example are the same as in the previous example, with the exception that the target proteins differ. The target proteins used here are modified versions of GFP while in the previous example they were mutant versions of YFP.

To generate data described in FIG. 5B, cells were grown in 500 nM aTc to induce expression of the target protein fused to Fcy-1 in SC-U media to observe mitochondria and SC-LU media to observe ER and peroxisome overnight. Then, the cells were grown to log phase in those media respectively. Cell images were acquired using R4 Revolve Fluorescence Microscope (Discover Echo). GFP fluorescence was detected using GFP filter. Mitochondria was stained with Mito Tracker Red FM (Thermo Fisher Scientific, M22425) for 1 hour and detected using RFP filter. ER was detected by using mCherry-Sec12 using RFP filter. Peroxisome was detected by using Pex11-mCherry using RFP filter.

### Example 3

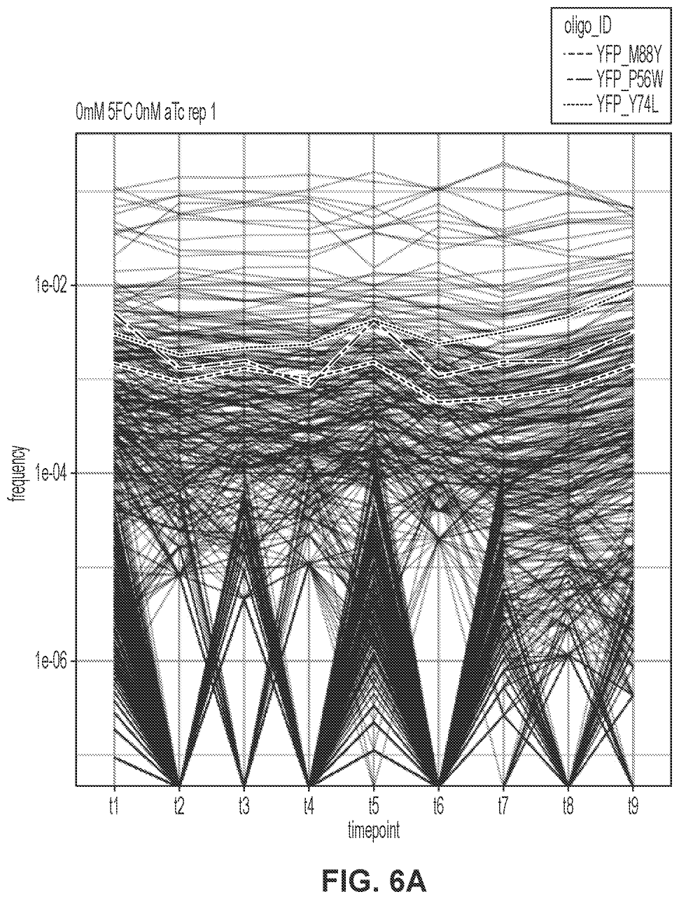

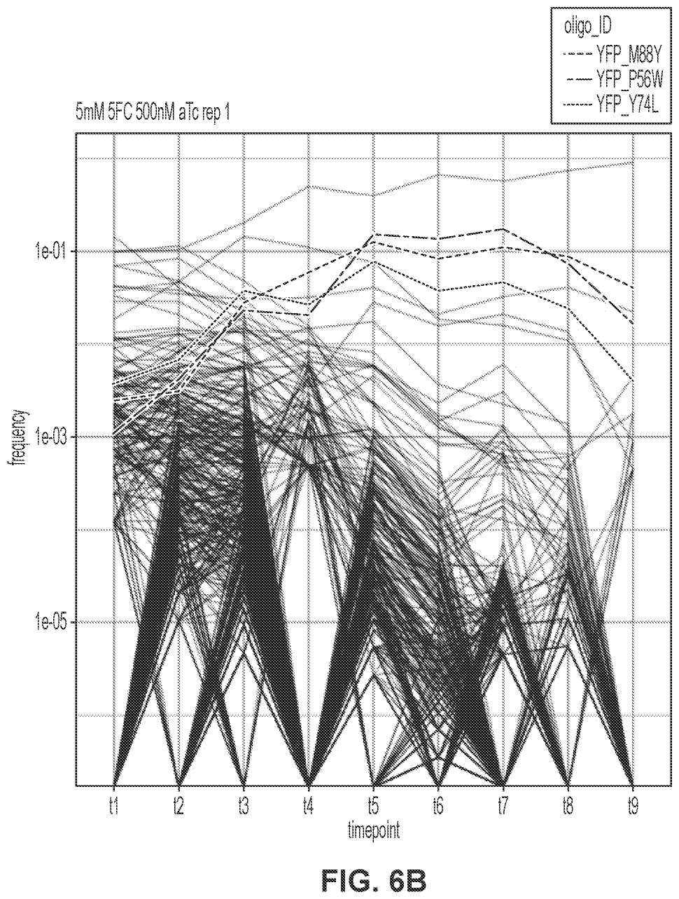

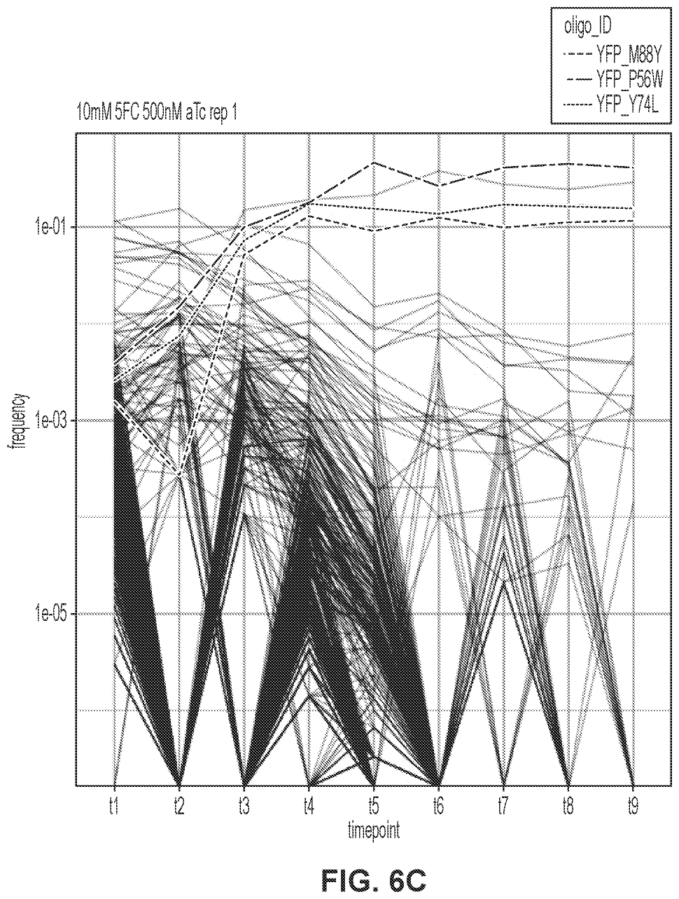

In some embodiments, the present disclosure builds off the idea that one can study a target protein's stability by anchoring it to an indicator protein that affects growth rate. This approach can be low throughput when growth is measured via some techniques. The present example took the approach of including a DNA barcode that identifies different variants of the target protein. This allows hundreds or thousands of different variants of the target protein to be combined in the same vessel and their relative fitness tracked by monitoring the frequency of their barcodes over time using next generation sequencing. In some embodiments, a control strain containing a wildtype version of the target protein can be included to serve as a benchmark of protein stability. This was not included in the current example. In the current example, about 200 different variants of YFP were expressed in yeast cells and studied using the method of the current disclosure. These variants have roughly equal fitness in conditions where the Fcy-1-YFP fusions are not expressed (OnM aTc) (FIG. 6A). But when these fusion proteins are expressed (500 nM aTc) and 5-FC is added to the media at either 5 mM (FIG. 6B) or 10 mM (FIG. 6C) most of the strains die and their barcodes fall to very low frequencies. Only target proteins that contain amino acid changes suspected to cause severe misfolding (dashed lines in FIG. 6) rise to higher frequency.

In this example, in order to count the relative frequencies of each strain and how they changed over time, the unique barcode region from each strain was prepared for sequencing. First, billions of yeast cells were sampled from the pooled competitive growth experiment every 24 hours. To extract their barcode-containing plasmids, these yeast were centrifuged at 15,000 rpm for 1 min. After removal of the supernatant, 250 μl of yeast lysis solution 1 (0.1 M Na₂EDTA, 1 M sorbitol, and pH 7.5) and 1 μl of Zymolyase at 5U/μl (Zymo Research, E1005) were added to the pellet. The sample was incubated at 37° C. for 30 min. After incubation, 250 μl of solution 2 (0.2 M NaOH, and 1% SDS) was added to the lysed sample and vortexed. Then, 250 μl of solution 3 (8.7% acetic acid and 5 M potassium acetate) was added and vortexed. After vortexing, the sample was centrifuged at 15,000 rpm for 10 min. 750 μl of the supernatant was transferred to the spin column included in Monarch Plasmid Miniprep Kit (New England BioLabs, T1010L), and the column was centrifuged at 13,000 rpm for 1 min. After discarding the flow-through, 200 μl of Plasmid Wash Buffer 1 included in the kit was added to the column, and the column was centrifuged at 13,000 rpm for 1 min. After discarding the flow-through, 400 μl of Plasmid Wash Buffer 2 included in the kit was added to the column, and the column was centrifuged at 13,000 rpm for 1 min. After discarding the flow-through, the column was spun at 13,000 rpm for 1 min for the removal of wash buffer completely. The column was inserted into a new 1.5 ml tube, and 30 μl of DNA Elution Buffer included in the kit was added to the center of the matrix on the column. After waiting 1 min at room temperature, the tube was centrifuged at 13,000 rpm for 1 min to elute plasmids. The concentration of the plasmid was quantified by using Qubit dsDNA HS Assay Kit (Thermo Fisher Scientific, Q32854) on Qubit 4 Fluorometer (Thermo Fisher Scientific, Q33226).

From the extracted plasmids, POR amplification of the barcode was performed by a two-step PCR scheme similar to the protocol described in Levy et al PMID 25731169 and Kinsler et al PMID 33263280. The forward and reverse primers which were used in the first PCR each had a unique 8-mer index for multiplexing in downstream analysis. For the first step of the two-step PCR, the one reaction consisted of 13 μl of Nuclease free H₂O, 10 μl of an extracted plasmid containing about 20 ng, 1 μl each of 10 μM forward and 10 μM reverse primer, and 25 μl of Hot Start Taq 2× Master Mix (New England BioLabs, M0496L). The first PCR was performed in hot-start PCR following the cycles: 1 cycle for 10 min at 94° C., 3 cycles for 3 min at 94° C.; 1 min at 55° C.; 1 min at 68° C., 1 cycle for 1 min at 68° C., and hold at 4° C. After the first PCR, the PCR product was cleaned up by using Monarch PCR & DNA Cleanup Kit (New England BioLabs, T1030L) following the manufacturer's protocol, and the cleaned-up PCR product was eluted in 22 μl. For the second PCR, the one reaction consisted of 14.5 μl of Nuclease free H₂O, 20 μl of a cleaned-up PCR product, 10 μl of 5× Q5 Reaction Buffer, 2 μl each of forward and reverse primer of Illumina index primers, 1 μl of 10 mM dNTPs (Thermo Fisher Scientific, 18427088), 0.5 μl of Q5 Hot Start High-Fidelity DNA Polymerase (New England BioLabs, M0493L). The second PCR was performed in hot-start PCR following the cycles: 1 cycle for 30 sec at 98° C., 2 cycles for 10 sec at 98° C.; for 20 sec at 69° C.; for 30 sec at 72° C., 2 cycles for 10 sec at 98° C.; for 20 sec at 67° C.; for 30 sec at 72° C., 20 cycles for 10 sec at 98° C.; for 20 sec at 65° C.; for 30 sec at 72° C., 1 cycle for 3 min at 72° C., and hold at 4° C. The whole PCR product was loaded onto 2% of NuSieve 3:1 Agarose (LONZA, 50090), and the band between 300 bp and 400 bp were sliced. The selected PCR product was extracted by Monarch DNA Gel Extraction Kit (New England BioLabs, T1020L) following the manufacturer's protocol, and the extracted PCR product was eluted in 10 μl. The concentration of the product was quantified by using Qubit dsDNA HS Assay Kit on Qubit 4 Fluorometer.

The resulting samples, each pertaining to a different timepoint or a different experiment where different concentrations of either aTc and 5FC were used, were multiplexed such that no two had similar Illumina or internal 8-mer indices, following a scheme to exclude any index swapping events that happened during NGS sequencing (Kinsler et al PMID 33263280). They were sequenced on either a Novoseq or a Hiseq X. Since these amplicons libraries have low diversity, we spiked in 20% genomic DNA to all sequencing runs.

To process the resulting sequencing data and infer changes in barcode freuenies over time, STAR index files were generated from YFP reference sequences by using STAR aligner Dobin et al PMID 32104886 with the following STAR commands; STAR--runMode genomeGenerate--runThreadN 10--genomeDir “STAR index output directory”--genomeFastaFiles “reference sequence FASTA file” --genomeSAindexNbases 8. NGS sequencing data were demultiplexed into mate-pair files, a forward mate read1 (R1) file and a reverse mate read2 (R2) file, by Illumina sequencer software following an 15 and 17 indexes in an Illumina adaptor sequence. To exclude PCR duplicates in downstream processing, the UMIs of R1 and R2 files were extracted by using UMI-tools (Smith et al PMID 28100584) with the following UMI-tools commands; umi_tools extract-I “R1 file”--bc-pattern=NNNNNN-S “extracted R1 output file”--read2-in= “R2 file”--bc-pattern2=NNNNNN--read2-out= “extracted R2 output file”. Then, the extracted R1 and R2 files were demultiplexed and trimmed 5′ end region containing the index by using FLEXBAR (Dodt et al PMID 24832523 and Roehr et al PMID 28541403) with the following FLEXBAR commands; flexbar-r “extracted R1 file”-p “extracted R2 file”-b “index FASTA file for R1”-b2 “index FASTA file for R2”-bt LEFT-be 0.125-n 10. The reads in the demultiplexed R1 and R2 files were aligned to the STAR index sequences with the following STAR commands; STAR--genomeDir “STAR index output directory”--readFilesin “demultiplexed R1 file” “demultiplexed R2 file”--runThreadN 10--outSAMtype BAM Unsorted--peOverlapNbasesMin 62--peOverlapMMp 0--outFilterMultimapNmax 1-outFilterMismatchNmax 0-alignEnds Type EndToEnd-alignintronMax 1--alignIntronMin 2--scoreDelOpen-10000--scoreInsOpen-10000--outFilterMatchNmin 137--alignSoftClipAtReferenceEnds No--outReadsUnmapped Fastx. The generated aligned sequence BAM file was sorted and indexed by using SAMtools Li et al MOID 19505943 with the following SAMtools commands; samtools sort-@ 8-o “sorted output BAM file” “unsorted output BAM file”, samtools Index “sorted BAM file”. The duplicated reads in the indexed BAM file were excluded by using UMI-tools with the following UMI-tools commands; umi_tools dedup-I “indexed BAM file”--paired-S “output BAM file without duplicated reads”--chimeric-pairs=discard--unpaired-reads=discard--method cluster. The mapped reads in the BAM file without duplicated reads were counted by using SAMtools with the following SAMtools commands; samtools index “BAM file without duplicated reads”, samtools idxstats “indexed BAM file without duplicated reads”> “indexed SAM file without duplicated reads”. After each barcode was counted, we plotted its frequency over the total number of barcodes over time to create the panels in FIG. 6.

Some further aspects are defined in the following clauses:

Clause 1: A method of detecting a misfolded target protein. The method comprising: determining a growth rate or relative fitness of at least a first cell population that comprises a first fusion polypeptide comprising a first variant of a target protein disposed between segments of a toxic indicator protein, which segments together induce the first cell population to grow at a lower rate when the target protein is relatively less misfolded than when the target protein is relatively more misfolded; and determining that the growth rate or fitness of the first cell population varies from a growth rate or fitness of at least a second cell population that comprises a second fusion polypeptide comprising a second variant of the target protein disposed between the segments of the toxic indicator protein, thereby detecting the misfolded target protein.

Clause 2: The method of Clause 1, further comprising quantifying a misfolding or stability measure of the first variant of the target protein by comparing the growth rate or fitness of the first cell population to that of at least one other cell population.

Clause 3: The method of Clause 1 or Clause 2, wherein the first cell population growth rate or fitness is higher or lower than the growth rate or fitness of the second cell population.

Clause 4: The method of any one of the preceding Clauses 1-3, wherein the target protein is attached to the segments of the toxic indicator protein via a linker moiety.

Clause 5: The method of any one of the preceding Clauses 1-4, wherein the second variant of the target protein is a wild-type form of the target protein.

Clause 6: The method of any one of the preceding Clauses 1-5, wherein the first variant of the target protein comprises one or more mutations.

Clause 7: The method of any one of the preceding Clauses 1-6, wherein the toxic indicator protein is an inducible toxic indicator protein.

Clause 8: The method of any one of the preceding Clauses 1-7, further comprising exposing the inducible toxic indicator protein to an inducing agent.

Clause 9: The method of any one of the preceding Clauses 1-8, wherein the inducible toxic indicator protein comprises an FCY1 protein and wherein the method further comprises contacting the first cell population with 5-fluorocytosine (5FC) prior to and/or when determining the growth rates of the first and second cell populations.

Clause 10: The method of any one of the preceding Clauses 1-9, comprising determining growth rates of multiple cell populations substantially in parallel with one another, wherein two or more of the multiple cell populations comprise different fusion polypeptides comprising different variants of the target protein disposed between the segments of the toxic indicator protein, and determining whether the growth rates or fitness of multiple cell populations vary from each other.

Clause 11: The method of any one of the preceding Clauses 1-10, wherein the different fusion polypeptides comprise about 3, about 4, about 5, about 6, about 7, about 8, about 9, about 10, about 20, about 30, about 40, about 50, about 60, about 70, about 80, about 90, about 100, about 200, about 300, about 400, about 500, about 600, about 700, about 800, about 900, about 1000, about 2000, about 3000, about 4000, about 5000, or more different variants of the target protein.

Clause 12: The method of any one of the preceding Clauses 1-11, further comprising generating nucleic acid variants that encode the different variants of the target protein.

Clause 13: The method of any one of the preceding Clauses 1-12, further comprising expressing the first and second fusion polypeptides from nucleic acid plasmids disposed in the first and second cell populations, which nucleic acid plasmids encode the first or the second fusion polypeptides.

Clause 14: The method of any one of the preceding Clauses 1-13, wherein the nucleic acid plasmids comprise one or more nucleic acid barcodes that distinguish a nucleic acid plasmid encoding the first fusion polypeptide from a nucleic acid plasmid encoding the second fusion polypeptide.

Clause 15: The method of any one of the preceding Clauses 1-14, wherein at least one of the nucleic acid barcodes comprises a randomly selected sequence of nucleotides.

Clause 16: The method of any one of the preceding Clauses 1-15, wherein the nucleic acid barcodes comprise donor and/or guide nucleic acid sequences and wherein the method further comprises using the nucleic acid barcodes that comprise the donor and/or the guide nucleic acid sequences to generate one or more mutations in a nucleic acid that encodes the target protein.

Clause 17: The method of any one of the preceding Clauses 1-16, comprising pooling the first and second cell populations in a container.

Clause 18: The method of any one of the preceding Clauses 1-17, comprising determining the growth rate or relative fitness of the first cell population and the growth rate or relative fitness of the second cell population from changes in nucleic acid barcode frequencies observed over time.

Clause 19: The method of any one of the preceding Clauses 1-18, wherein the target protein comprises a disease-associated protein.

Clause 20: The method of any one of the preceding Clauses 1-19, wherein the target protein comprises a candidate therapeutic protein.

Clause 21: The method of any one of the preceding Clauses 1-20, wherein the target protein comprises a recombinantly engineered protein.

Clause 22: The method of any one of the preceding Clauses 1-21, wherein the first variant and/or the second variant of the target protein is disposed between the segments of the toxic indicator protein such that localization of the target protein is at least disrupted.

Clause 23: A nucleic acid plasmid, comprising a nucleotide sequence that encodes a fusion polypeptide comprising a variant of a target protein disposed between segments of a toxic indicator protein, wherein upon expression of the fusion polypeptide in a cell population, the segments together induce the cell population to grow at a lower rate when the target protein is relatively less misfolded than when the target protein relatively more misfolded.

Clause 24: The nucleic acid plasmid of Clause 23, further comprising additional nucleotide sequences disposed between the segments of the toxic indicator protein and the variant of the target protein, which additional nucleotide sequences encode polypeptide linker moieties.

Clause 25: The nucleic acid plasmid of Clause 23 or Clause 24, wherein the variant of the target protein is a wild-type form of the target protein.

Clause 26: The nucleic acid plasmid of any one of the preceding Clauses 23-25, wherein the variant of the target protein comprises one or more mutations.

Clause 27: The nucleic acid plasmid of any one of the preceding Clauses 23-26, wherein the toxic indicator protein is an inducible toxic indicator protein.

Clause 28: The nucleic acid plasmid of any one of the preceding Clauses 23-27, wherein the inducible toxic indicator protein comprises an FCY1 protein.

Clause 29: The nucleic acid plasmid of any one of the preceding Clauses 23-28, wherein the target protein comprises a disease-associated protein.

Clause 30: The nucleic acid plasmid of any one of the preceding Clauses 23-29, wherein the target protein comprises a candidate therapeutic protein.

Clause 31: The nucleic acid plasmid of any one of the preceding Clauses 23-30, wherein the target protein comprises a recombinantly engineered protein.

Clause 32: The nucleic acid plasmid of any one of the preceding Clauses 23-31, wherein the variant of the target protein is disposed between the segments of the toxic indicator protein such that localization of the target protein is at least disrupted upon expression of the fusion polypeptide in the cell population.

Clause 33: A cell population comprising the nucleic acid plasmid of any one of the preceding Clauses 23-32.

Clause 34: A kit comprising the nucleic acid plasmid of any one of the preceding Clauses 23-33.

Clause 35: A system, comprising: a sample container positioning area that comprises a sample container that comprises at least a first cell population that comprises a first fusion polypeptide comprising a first variant of a target protein disposed between segments of a toxic indicator protein, which segments together induce the first cell population to grow at a lower rate when the target protein is relatively less misfolded than when the target protein relatively more misfolded; a detector configured to detect a growth rate or fitness of the first cell population; and, a controller that comprises, or is capable of accessing, computer readable media comprising non-transitory computer-executable instructions which, when executed by at least one electronic processor, perform at least: determining whether the growth rate or fitness of the first cell population varies from a growth rate or fitness of at least a second cell population that comprises a second fusion polypeptide comprising a second variant of the target protein disposed between the segments of the toxic indicator protein.

Clause 36: The system of Clause 35, wherein the inducible toxic indicator protein comprises an FCY1 protein.

Clause 37: The system of Clause 35 or Clause 36, wherein the first fusion polypeptide is expressed from nucleic acid plasmids disposed in the first cell population, which nucleic acid plasmids encode the first fusion polypeptide and comprise one or more nucleic acid barcodes that distinguish the nucleic acid plasmid encoding the first fusion polypeptide from a nucleic acid plasmid encoding the second fusion polypeptide, wherein the detector comprises a nucleic acid sequencing device that generates sequence information from the nucleic acid plasmids, and wherein the non-transitory computer-executable instructions which, when executed by the electronic processor, further perform determining the growth rate of the first cell population and the growth rate of the second cell population from changes in nucleic acid barcode frequencies observed over time in the sequence information.

Clause 38: The system of any one of the preceding Clauses 35-37, wherein the first fusion polypeptide is expressed from a set of first nucleic acid plasmids disposed in the first cell population, which first nucleic acid plasmids encode the first fusion polypeptide, and wherein a set of second nucleic acid plasmids disposed in the first cell population comprise one or more nucleic acid barcodes that distinguish the first nucleic acid plasmid encoding the first fusion polypeptide from a third nucleic acid plasmid encoding the second fusion polypeptide, wherein the detector comprises a nucleic acid sequencing device that generates sequence information from the nucleic acid plasmids, and wherein the non-transitory computer-executable instructions which, when executed by the electronic processor, further perform determining the growth rate of the first cell population and the growth rate of the second cell population from changes in nucleic acid barcode frequencies observed over time in the sequence information.

Clause 39: The system of any one of the preceding Clauses 35-38, wherein at least one of the nucleic acid barcodes comprises a randomly selected sequence of nucleotides.

Clause 40: The system of any one of the preceding Clauses 35-39, wherein the nucleic acid barcodes comprise donor and/or guide nucleic acid sequences that are used to generate one or more mutations in a nucleic acid that encodes the target protein.

Clause 41: The system of any one of the preceding Clauses 35-40, wherein the first variant and/or the second variant of the target protein is disposed between the segments of the toxic indicator protein such that localization of the target protein is at least disrupted.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

1. A method of detecting a misfolded target protein, the method comprising:
determining a growth rate or relative fitness of at least a first cell population that comprises a first fusion polypeptide comprising a first variant of a target protein disposed between segments of a toxic indicator protein, which segments together induce the first cell population to grow at a lower rate when the target protein is relatively less misfolded than when the target protein is relatively more misfolded; and,
determining that the growth rate or fitness of the first cell population varies from a growth rate or fitness of at least a second cell population that comprises a second fusion polypeptide comprising a second variant of the target protein disposed between the segments of the toxic indicator protein, thereby detecting the misfolded target protein.

2. The method of claim 1, further comprising quantifying a misfolding or stability measure of the first variant of the target protein by comparing the growth rate or fitness of the first cell population to that of at least one other cell population.

3.-6. (canceled)

7. The method of claim 1, wherein the toxic indicator protein is an inducible toxic indicator protein.

8. The method of claim 7, further comprising exposing the inducible toxic indicator protein to an inducing agent.

9. The method of claim 7, wherein the inducible toxic indicator protein comprises an FCY1 protein and wherein the method further comprises contacting the first cell population with 5-fluorocytosine (5FC) prior to and/or when determining the growth rates of the first and second cell populations.

10. The method of claim 1, comprising determining growth rates of multiple cell populations substantially in parallel with one another, wherein two or more of the multiple cell populations comprise different fusion polypeptides comprising different variants of the target protein disposed between the segments of the toxic indicator protein, and determining whether the growth rates or fitness of multiple cell populations vary from each other.

11. (canceled)

12. (canceled)

13. The method of claim 1, further comprising expressing the first and second fusion polypeptides from nucleic acid plasmids disposed in the first and second cell populations, which nucleic acid plasmids encode the first or the second fusion polypeptides.

14.-22. (canceled)

23. A nucleic acid plasmid, comprising a nucleotide sequence that encodes a fusion polypeptide comprising a variant of a target protein disposed between segments of a toxic indicator protein, wherein upon expression of the fusion polypeptide in a cell population, the segments together induce the cell population to grow at a lower rate when the target protein is relatively less misfolded than when the target protein relatively more misfolded.

24.-26. (canceled)

27. The nucleic acid plasmid of claim 23, wherein the toxic indicator protein is an inducible toxic indicator protein.

28. The nucleic acid plasmid of claim 27, wherein the inducible toxic indicator protein comprises an FCY1 protein.

29.-31. (canceled)

32. The nucleic acid plasmid of claim 23, wherein the variant of the target protein is disposed between the segments of the toxic indicator protein such that localization of the target protein is at least disrupted upon expression of the fusion polypeptide in the cell population.

33. A cell population comprising the nucleic acid plasmid of claim 23.

34. A kit comprising the nucleic acid plasmid of claim 23.

35. A system, comprising:
a sample container positioning area that comprises a sample container that comprises at least a first cell population that comprises a first fusion polypeptide comprising a first variant of a target protein disposed between segments of a toxic indicator protein, which segments together induce the first cell population to grow at a lower rate when the target protein is relatively less misfolded than when the target protein relatively more misfolded;
a detector configured to detect a growth rate or fitness of the first cell population; and,
a controller that comprises, or is capable of accessing, computer readable media comprising non-transitory computer-executable instructions which, when executed by at least one electronic processor, perform at least:
determining whether the growth rate or fitness of the first cell population varies from a growth rate or fitness of at least a second cell population that comprises a second fusion polypeptide comprising a second variant of the target protein disposed between the segments of the toxic indicator protein.

36. The system of claim 35, wherein the inducible toxic indicator protein comprises an FCY1 protein.

37. The system of claim 35, wherein the first fusion polypeptide is expressed from nucleic acid plasmids disposed in the first cell population, which nucleic acid plasmids encode the first fusion polypeptide and comprise one or more nucleic acid barcodes that distinguish the nucleic acid plasmid encoding the first fusion polypeptide from a nucleic acid plasmid encoding the second fusion polypeptide, wherein the detector comprises a nucleic acid sequencing device that generates sequence information from the nucleic acid plasmids, and wherein the non-transitory computer-executable instructions which, when executed by the electronic processor, further perform determining the growth rate of the first cell population and the growth rate of the second cell population from changes in nucleic acid barcode frequencies observed over time in the sequence information.

38. The system of claim 35, wherein the first fusion polypeptide is expressed from a set of first nucleic acid plasmids disposed in the first cell population, which first nucleic acid plasmids encode the first fusion polypeptide, and wherein a set of second nucleic acid plasmids disposed in the first cell population comprise one or more nucleic acid barcodes that distinguish the first nucleic acid plasmid encoding the first fusion polypeptide from a third nucleic acid plasmid encoding the second fusion polypeptide, wherein the detector comprises a nucleic acid sequencing device that generates sequence information from the nucleic acid plasmids, and wherein the non-transitory computer-executable instructions which, when executed by the electronic processor, further perform determining the growth rate of the first cell population and the growth rate of the second cell population from changes in nucleic acid barcode frequencies observed over time in the sequence information.

39. The system of claim 37, wherein at least one of the nucleic acid barcodes comprises a randomly selected sequence of nucleotides.

40. The system of claim 37, wherein the nucleic acid barcodes comprise donor and/or guide nucleic acid sequences that are used to generate one or more mutations in a nucleic acid that encodes the target protein.

41. The system of claim 35, wherein the first variant and/or the second variant of the target protein is disposed between the segments of the toxic indicator protein such that localization of the target protein is at least disrupted.
